(19) United States
(12) Patent Application Publication
(10) Pub. No.: US 2011/0136176 A1
(43) Pub. Date: Jun. 9, 2011

(54) METHOD FOR TREATING LIGNOCELLULOSE RAW MATERIAL

(75) Inventors: Takafumi SHIMODA, Minamiuonuma-shi (JP); Kozo NISHIBORI, Tokamachi-shi (JP); Yosuke BABA, Minamiuonuma-shi (JP)

(73) Assignee: YUKIGUNI MAITAKE CO., LTD., Minamiuonuma-shi (JP)

(21) Appl. No.: 13/021,417

(22) Filed: Feb. 4, 2011

Related U.S. Application Data
(63) Continuation of application No. PCT/JP2009/063919, filed on Aug. 6, 2009.

(30) Foreign Application Priority Data
Aug. 7, 2008 (JP) 2008-204143

(51) Int. Cl. C12P 19/00

(52) U.S. Cl. 435/72

(57) ABSTRACT

A method for treating a lignocellulose raw material so as to facilitate a pulverization treatment that is necessary for the efficient and effective use of a lignocellulose raw material as a raw material or resource for material conversion. When converting a lignocellulose raw material into sugar or a useful material, such as ethanol, with the aid of an enzyme or producing a biodegradable material derived from a lignocellulose raw material via a mechanical or chemical treatment, a lignocellulose raw material is treated with an enzyme prior to or simultaneously with the pulverization process, so that the viscosity of slurry comprising a lignocellulose raw material and water is lowered and the pulverization efficiency is improved.

### CROSS REFERENCE TO RELATED APPLICATION

This is a continuation application of International Application No. PCT/JP2009/063919, filed on Aug. 6, 2009, which claimed the priority of Japanese Application No. 2008-204143, filed Aug. 7, 2008, the entire content of each of which is incorporated herein by reference.

### TECHNICAL FIELD

The present invention relates to a method that is conducted when converting substances via an enzyme reaction alone or in combination with the use of microorganisms, so as to obtain lignocellulose raw materials and useful substances from lignocellulose raw materials comprising wastes of the former lignocellulose raw materials.

### BACKGROUND ART

Wood materials have heretofore been used for buildings, paper, tools, and the like, and a great deal of research has been conducted regarding applications thereof. Lignocellulose raw materials contained in wood materials are resources that are abundant on the earth, and expansion of the applications thereof as so-called biomass resources has been desired.

Carbon contained in wood materials is carbon dioxide in the air that is absorbed and fixed by plants. Such carbon is offset by regenerating plants that absorb carbon dioxide in an amount equivalent to the amount of carbon dioxide generated when producing energy. Accordingly, research has been conducted on the use of wood materials for the purpose of the creation of a recycling society and research has been made on techniques of utilizing waste wood materials as fuels. For example, research has been conducted on techniques of utilizing waste wood materials for fuels, such as wood material-derived fuels that are obtained by treating waste wood materials and can be used for cement kilns or boilers and a method for producing such fuels (JP Patent No. 3929371) and a novel recycling process via combustion of waste wood materials treated with CCA (i.e., copper, chromium, and arsenic) (JP Patent No. 3727067).

In addition, a method for producing pulp comprising steaming woody biomass in the presence of ammonia or an ammonia generator for the purpose of producing biodegradable sheets or biodegradable vessels selectively from woody biomass (JP Patent Publication (kokai) No. 2007-002383); i.e., a method of effectively using woody biomass for variety of materials, has been studied. Also, an attempt of converting lignocellulose raw materials into other useful substances, such as ethanol or polylactic acid, has been made with the use of enzymes or microorganisms (Japan Institute of Energy, 2002, Biomass Handbook, pp. 152-197). When converting lignocellulose raw materials into ethanol to prepare liquid fuels, for example, cellulose in lignocellulose may be degraded with an enzyme and fermented by yeast. Cellulose in lignocellulose, however, is surrounded by lignin or hemicellulose, and crystallinity thereof is high. This disadvantageously makes degradation of cellulose by a cellulose-degrading enzyme (cellulase) difficult.

In order to utilize cellulose in lignocellulose raw materials, accordingly, lignocellulose needs to be modified for ease of use by, for example, reducing the crystallinity of cellulose or removing lignin. That is, some kind of pre-treatment is necessary before an enzyme reaction so as to improve the hydrolyzability of the cellulose portion (Shiro Saka et al., 2001, Biomass, Energy, Environment, IPC, pp. 251-260; Jun Sugiura, 2002, Biomass energy characteristics and technology of energy conversion and use, NTS, pp. 283-312; and George P. Philippidis, 1996, Handbook on Bioethanol, Taylor &Francis, pp. 253-285).

A fine pulverization method is a method wherein a biomass is formed into fine particles with the use of a pulverizing device such as a ball mill, so as to increase the surface area of cellulose contained in the biomass to thereby facilitate cellulose degradation. In such a case, it is said that the particle size should be minimized (Merill A. Millet, et al., 1976, Biotechnol. & Bioeng. Symp., No. 6, pp. 125-153).

When production of materials for biodegradable vessels from woody biomass is intended as well as conversion thereof into fuel substances such as ethanol, lignocellulose raw materials are subjected to a mechanical or chemical treatment to produce cellulose fiber aggregates that serve as raw materials. Pulverization of lignocellulose raw materials is advantageous.

Examples of pulverizing devices include bead mills and disc mills. When pulverization is continuously carried out with the use of bead mills or other means, however, the slurry viscosity must be taken into consideration when slurry comprising a lignocellulose raw material and water is proceeded to the next treatment process with the aid of a pump.

Some biomasses have high slurry viscosity, such biomasses clog the pump, and transition to the next process of pulverization is disturbed. Even if transition proceeds, disadvantageously, pulverization efficiency is low and a pulverizing device becomes clogged.

### SUMMARY OF THE INVENTION

### Object to be Attained by the Invention

It is an object of the present invention to develop a method for treating a lignocellulose raw material so as to facilitate a pulverization treatment that is necessary for the efficient and effective use of a lignocellulose raw material as a raw material or resource for material conversion.

### Means for Attaining the Object

The present inventors have conducted concentrated studies in order to attain the above object. As a result, they discovered that, when converting a lignocellulose raw material into sugar or a useful material, such as ethanol, with the aid of an enzyme or producing a biodegradable material derived from a lignocellulose raw material via a mechanical or chemical treatment, a lignocellulose raw material may be treated with an enzyme prior to or simultaneously with the pulverization process, so that the viscosity of slurry comprising a lignocellulose raw material and water would be lowered and the pulverization efficiency would be improved. This has led to the completion of the present invention.

Specifically, the present invention concerns a method for obtaining a final product in good yield by treating a lignocellulose raw material with an enzyme prior to pulverization of a lignocellulose raw material. The method is described in detail.

The present invention relates to the following.

(1) A method for treating a lignocellulose raw material comprising performing an enzyme treatment prior to pulverization of a lignocellulose raw material so as to lower the viscosity of the lignocellulose raw material and improve the pulverization efficiency.

(2) A method for treating a lignocellulose raw material comprising performing an enzyme treatment simultaneously with pulverization of a lignocellulose raw material so as to lower the viscosity of the lignocellulose raw material and improve the pulverization efficiency.

(3) The method for treating a lignocellulose raw material according to (1) or (2), wherein the enzyme used for treating the lignocellulose raw material is cellulase.

(4) The method for treating a lignocellulose raw material according to any of (1) to (3), wherein the lignocellulose raw material is a waste mushroom bed.

A lignocellulose raw material used for the treatment method may be in a dry or wet state and the moisture condition thereof is not limited. The size of a biomass is not limited as long as it can be introduced into a vessel used for the reaction. By reducing the size thereof, however, the reaction can be accelerated.

Examples of lignocellulose raw materials that can be used include: any herbaceous lignocellulose raw materials, such as rice straw, rice husk, wheat straw, bagasse, any part of maize, or a different type of plant such as switchgrass; and any woody lignocellulose raw materials, such as softwood or hardwood chips, wood thinnings, construction debris, or a waste mushroom bed. Further, used paper, cotton, or the like can be used.

A lignocellulose raw material, water or buffer, and an enzyme may be mixed in a vessel, and the resulting mixture may be allowed to react for 30 to 90 minutes, so that the viscosity of lignocellulose slurry would be lowered with the elapse of time. A vessel made of any material such as plastic, stainless steel, iron, or a different metal can be used.

Examples of enzymes that can be used for treatment include cellulase, hemicellulase, glucanase, glucosidase, amylase, glucoamylase, laccase, manganese peroxidase, lignin peroxidase, and a mixture of some or all of such enzymes. Use of cellulase can produce the best results. An enzyme may be a commercially available enzyme, a culture solution used for culture of filamentous bacteria, or an enzyme purified therefrom, provided that such enzyme is applicable to an intended purpose. When cellulase is used, for example, a commercially available enzyme or roughly-purified enzyme often contains both cellulase and hemicellulase.

The amount of the enzyme used may be adequately determined in accordance with its type. It is effective to acid hemicellulase-containing cellulase in amounts of 50 FPU (Filter Paper Unit, filter paper degradation activity) per waste mushroom bed. Also, a lignin-degrading enzyme may be introduced as another enzyme to degrade lignin, and hemicellulase, such as xylanase, can be used. When preparing an enzyme solution, an enzyme may be suspended in water. It is also effective to maintain the pH at 4 to 5 with the use of acetate or citrate buffer. An enzyme solution can be prevented from bacterial contamination by removing bacteria through a filter with a size of 0.45 μm or less.

An enzyme solution may be allowed to stand still when an enzyme treatment is carried out. By agitating the enzyme solution with the use of an agitator or other means, the degree of contact between an enzyme and a substrate is increased. While a reaction at room temperature can produce satisfactory effects, an enzyme reaction is accelerated by maintaining temperature optimal for an enzyme of interest.

An enzyme treatment is preferably carried out prior to pulverization, although it may be simultaneously carried out.

A lignocellulose raw material having a viscosity lowered by such enzyme treatment is supplied to various pulverizing devices manually or automatically with the use of a pump or other means and then subjected to pulverization. When the material is automatically supplied, the slurry viscosity is lowered, and transportation via a pump can then be easily carried out. Pulverizing devices to be used may be selected in accordance with the intended applications, and wet-type continuous pulverizing devices, such as ball mills, bead mills, disc mills, mortar mills, or high-shear mills, are preferable. This enzyme treatment enables pulverization of slurry that would clog a pulverizing device when it is not subjected to treatment. As the duration of the slurry of the lignocellulose raw material remaining in a pulverizing device is prolonged, an interaction results from a physical treatment using a pulverizing device and a chemical treatment via an enzyme reaction, and the pulverization effects are further improved.

This treatment method not only produces a useful substance such as ethanol from a lignocellulose raw material, but also enables the use of a lignocellulose raw material as a raw material for a lignocellulose-based product, such as a fiberboard, particle board, plywood, or paper. In addition, a lignocellulose raw material may be carbonized and used as a carbonized material, such as char or activated carbon. Further, it may be converted into a pellet, a film, or biodegradable solid material in accordance with the intended purpose. That is, any material comprising lignocellulose as a raw material can be produced. This treatment method is applicable to methods for producing any materials, such as a method for producing a material, such as a biodegradable plastic, carbonized material, or paper, from a lignocellulose raw material, provided that such methods involve pulverization.

This description includes part or all of the contents as disclosed in the description and/or drawings of Japanese Patent Application No. 2008-204143, which is a priority document of the present application.

### Effects of the Invention

According to the present invention, a lignocellulose raw material is treated with an enzyme and pulverized prior to or simultaneously with conversion of a lignocellulose raw material into another material, in particular, conversion thereof into a sugar using an enzyme and into ethanol using microorganisms. This lowers the viscosity of the lignocellulose raw material, prevents the problem of clogging of a pulverizing device, and consequently improves the pulverization efficiency.

### BRIEF DESCRIPTION OF THE DRAWINGS

FIG. 1 shows an influence of the present invention on the viscosity of the waste mushroom bed slurry.

FIG. 2 shows an influence of the present invention on the ethanol yield after pulverization.

### BEST MODES FOR CARRYING OUT THE INVENTION

The present invention is hereafter described in greater detail with reference to the following example, although the technical scope of the present invention is not limited thereto.

### Example

### Effects of the Invention on Ethanol Conversion with the Use of a Maitake Waste Mushroom Bed

(1) Effects of the Invention on Ethanol Conversion with the Use of a Maitake Waste Mushroom Bed

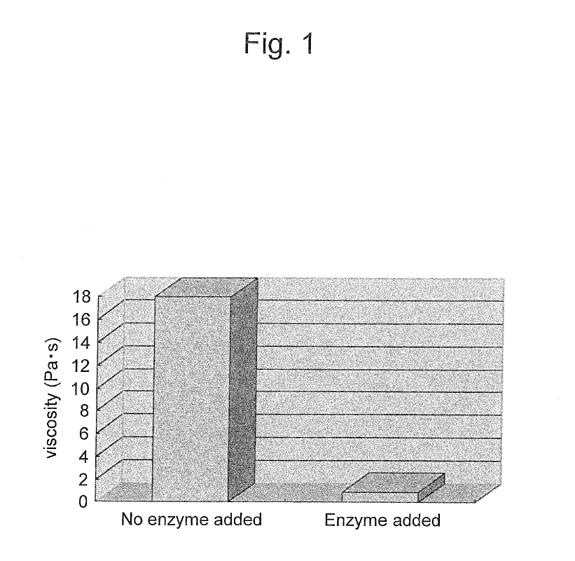

An example of the practice of the present invention with the use of a maitake waste mushroom bed, which is a woody biomass, as a cellulose-based biomass is described below. A maitake waste mushroom bed substantially consists of hardwood sawdust with a moisture content of 60% or more. Such waste mushroom beds (15 kg) were introduced into a stainless vessel used as a reaction vessel, and 27 liters of tap water and 0.65 kg of powder cellulase (GODO-TCD, Godo Shusei Co., Ltd.) were added thereto. The reaction was allowed to proceed at room temperature for 90 minutes while agitating the solution with the use of the Dissolver (DHV-5, Inoue Manufacturing Co., Ltd.). A control reaction was carried out without the addition of an enzyme. The viscosity of the waste mushroom bed slurry 90 minutes after the initiation of the reaction was measured using a viscometer. The results thereof are shown in FIG. 1. The viscosity of the slurry to which an enzyme had been added was lowered to 1/20 of the slurry to which an enzyme had not been added.

Glass beads (f: 2 to 2.8 mm) were filled in a vessel with a filling rate of 70%, the waste mushroom bed slurry after the reaction was supplied to the bead mills set at the peripheral speed of 14.5 m/s (Mighty Mill, MHG-II, Inoue Manufacturing Co., Ltd.) so as to set the discharge rate at 0.5 to 0.6 kg/m, and pulverization was carried out. The waste mushroom bed to which an enzyme had been added was treated without clogging the mills; however, the waste mushroom bed to which an enzyme had not been added clogged the vessel after a given amount thereof had been treated, and no further treatment was carried out (Table 1).

TABLE 1
Influence of the invention on clogging of bead mills

| | |
|---|---|
| Test group | Clogging of bead mills |
| Enzyme added | Occurred |
| No enzyme added | Not occurred |

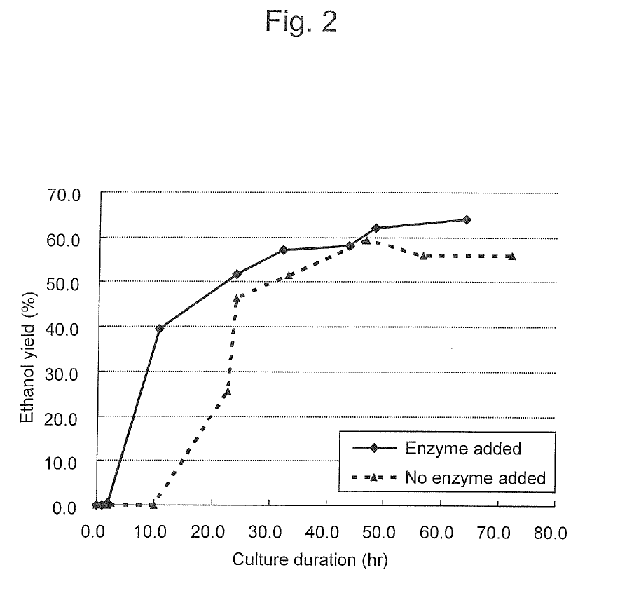

Subsequently, the pulverized culture product was introduced into a fermentation tank, baker's yeast was added at 1 g/l, and ethanol conversion was carried out at 30° C. To the control sample to which an enzyme had not been added, 0.65 kg of cellulase was added in addition to the yeast. Sampling was adequately carried out during the conversion reaction, and the ethanol concentration in the supernatant was measured via gas chromatography (GB-14; Shimadzu Corporation). The results are shown in FIG. 2. The ethanol yield is represented by the percentage of the amount of ethanol obtained with respect to the ideal amount of ethanol calculated based on the amount of cellulose in a waste mushroom bed. As a result, it was found that the ethanol yield obtained via conversion according to the method of the present invention was 1.1 times greater than that obtained via conversion without the addition of the enzyme.

All publications, patents, and patent applications cited herein are incorporated herein by reference in their entirety.

1. A method for treating a lignocellulose raw material comprising performing an enzyme treatment prior to pulverization of a lignocellulose raw material so as to lower the viscosity of the lignocellulose raw material and improve the pulverization efficiency.

2. A method for treating a lignocellulose raw material comprising performing an enzyme treatment simultaneously with pulverization of a lignocellulose raw material so as to lower the viscosity of the lignocellulose raw material and improve the pulverization efficiency.

3. The method for treating a lignocellulose raw material according to claim 1, wherein the enzyme used for treating the lignocellulose raw material is cellulase.

4. The method for treating a lignocellulose raw material according to claim 1, wherein the lignocellulose raw material is a waste mushroom bed.

5. The method for treating a lignocellulose raw material according to claim 2, wherein the enzyme used for treating the lignocellulose raw material is cellulase.

6. The method for treating a lignocellulose raw material according to claim 2, wherein the lignocellulose raw material is a waste mushroom bed.

7. The method for treating a lignocellulose raw material according to claim 3, wherein the lignocellulose raw material is a waste mushroom bed.
